(19) United States
(12) Patent Application Publication
(10) Pub. No.: US 2009/0030383 A1
(43) Pub. Date: Jan. 29, 2009

(54) Suction Method and a Wound Suction System

(76) Inventors: Truels Sterm Larsen, Frederiksberg (DK); Erik Lund Jepsen, Vaerlose (DE); Niels Hvid, Vedbaek (DE)

Correspondence Address: JACOBSON HOLMAN PLLC, 400 SEVENTH STREET N.W., SUITE 600, WASHINGTON, DC, 20004, US

(21) Appl. No.: 12/087,113

(22) PCT Filed: Feb. 1, 2007
(86) PCT No.: PCT/DK2007/000053

§ 371 Date: Jun. 26, 2008

(30) Foreign Application Priority Data
Feb. 2, 2006 (DK) PA 2006 00147
Feb. 2, 2006 (DK) PA 2006 00148
Feb. 2, 2006 (DK) PA 2006 00149
Jul. 24, 2006 (DK) PA 2006 01010

(51) Int. Cl. A61M 1/00

(52) U.S. Cl. 604/290; 604/315

(57) ABSTRACT

A suction system for removal of fluids from a surface, e.g. a wound site, the system comprising a cover (1) attachable to a circumference of the surface to form an enclosed suction space sealed from ambient space. The system further comprises a pump (4) in fluid communication with the enclosure via a tube (2) to provide a pressure difference between a suction pressure in the enclosure and an air pressure of an ambient space. To provide an improvement over the known systems and to facilitate pressure measurement, the system comprises flushing means (11) to clean the tube.

The present invention relates to a method for removal of fluids etc. Such a method could be applicable e.g. for wound treatment for enhancing wound healing. The invention further relates to a wound treatment suction system comprising a wound cover, which is attachable to a wound circumference of a living being to form an enclosed suction space. The system further comprises a pump which, via a tube, provides a pressure difference between a suction pressure in the suction space and an air pressure of an ambient space.

### BACKGROUND OF THE INVENTION

It has been found that fluid drainage of wounds promotes tissue growth and thereby facilitates a reduced healing time. The treatment has been exercised for many years and various therapeutic apparatus for providing suction to a wound have been developed.

U.S. Pat. No. 6,648,862 describes a vacuum desiccator using a canister which contains a trapping agent, and WO 97/18007 discloses a portable wound treatment apparatus with a canister and a pump arranged in a housing which promotes portable use, e.g. wearable on a harness or via a belt.

In WO 97/18007 is disclosed a system with a valve in fluid connection with the wound enclosure, said valve may be activated in order to relieve the pressure over the wound for a period of time. The valve is closed when the pump is operating.

In the known apparatuses, a wound cover is fixed in a sealing manner to the skin of a living being so that an enclosure is formed around the wound. The cover is connected to a pump, and suction is applied so a negative pressure over the wound is induced. The suction moves exudates and other contaminants from the enclosure to a receptacle, such as a canister for receiving the exudates. A filter may prevent contamination of the ambient space by the exhaust gas.

The pump generates a negative pressure that is applied to the wound. As the pump is located remote to the wound and is connected to the wound enclosure by a tube, it may be difficult to control the exact pressure over the wound, as the tube from time to time may be filled with liquid or slough from the wound, which will affect the pressure in the system. Thus the negative pressure applied by the pump may deviate from the negative pressure over the wound. In the following, negative pressure denotes a pressure which is lower than a reference pressure, and the reference pressure is typically a pressure of the surrounding atmosphere.

### DESCRIPTION OF THE INVENTION

It is objects of embodiments of the invention to:
ensure correct measurement of pressure in a negative pressure system e.g. for wound healing.
achieve and maintain a well-defined negative pressure over a treated surface.
avoid clotting of the tube between the surface and the pump substantially without affecting the pressure over the surface.
provide a less power consuming system for removing fluid by suction.

Accordingly, the invention, in a first aspect, provides a method for removing fluid from a surface, the method comprising:
providing a cover which is attachable to a circumference of the surface to form an enclosed suction space separate from an ambient space,
providing a first tube forming a flow path between an inlet located in the suction space and an outlet located outside the suction space,
providing, by suction via the first tube, a pressure difference between a suction pressure in the suction space and an ambient pressure in the ambient space, and
flushing the first tube by providing, in a first period of time, a flow of a flushing fluid through at least a major part of the tube.

The fluid which is removed from the surface by suction could be a mixture of liquid and gas. When this mixture enters the first tube, the liquid may fill the tube and form a liquid string, and thus block the free passage of gas through the tube. This may influence the pressure in the enclosure as well as measurement of the pressure, and it may cause fluctuations in the pressure over the surface. If the surface is a wound surface, exudate from the wound may further be mixed with solid particles which, when located in the first tube, further increases the inaccuracy of the pressure and measurement.

According to the present invention, this problem is solved by flushing the tube with a flushing fluid, e.g. by use of a first valve establishing communication between the tube and the ambient space. By providing a fluid flow through the tube, objects which are located in the tube can be flushed out of the tube and a more clean tube can be obtained. The flushed and therefore at least relatively clean tube enhances accuracy in measuring a pressure in the suction space through the tube, it increases the ability to set a pressure precisely, and it may reduce noise and energy consumption. By flushing in this context is meant that the flushing fluid is conducted in the direction away from the cover through the major part of the tube with intensity such that liquids and optionally also solid particles are removed from the flow path.

The tube could be a medical hose or any kind of tube, e.g. a plastic tube, e.g. in a length of 0.5 metre to 2.5 metre. The inlet may be attached to the cover, or the tube may penetrate the cover and extend into the suction space. In the opposite end of the tube, the outlet could be connected to the pump, e.g. via a releasably attached connector member.

The cover may form a cavity with an opening surrounded by an edge portion which facilitates an essentially gas tight connection to the surface, e.g. via a soft resilient adhesive material. The cover may be occlusive or semi-occlusive, e.g. being vapor permeable but water impermeable.

The flushing fluid could be atmospheric air, or it could be a gas, e.g. having sterilising or similar purification capabilities, and the flow could be provided between the inlet and the outlet or at least from a point which is close to the inlet, e.g. from a point which is in a distance of less than 10 pct of a total length of the tube from the inlet. The tube may be flushed by releasing an amount of flush air either into the suction space or into the first tube at a location near the inlet. The flush air could be released via a first valve which is arranged to control fluid communication from a container which contains the flushing fluid, or simply via a release passage between the ambient space and the suction space, or between the ambient space and the flow path. The first period of time may have a length which satisfies a flow through an entire length of the first tube. I.e. the first period of time is selected based on the length of the tube and the flow speed in the flow path. The valve may be opened for only as short a period as possible to provide just enough air flow to flush the entire tube and yet preserve a negative pressure in the suction space, or the first tube may be flushed during a longer period of time in which the pressure difference between the suction pressure and ambient pressure is checked so that the flushing can be stopped before the negative pressure in the suction space disappears.

The suction could be provided by use of a pump which is sufficiently strong to draw liquids away from the surface, e.g. a negative pressure in the range of 10 to 600 mm Hg. The negative pressure may be determined either relative to a reference pressure or as an absolute pressure, i.e. typically relative to a stiffness of a pressure sensing membrane or relative to a reference chamber in a pressure sensor. The pump could be activated in a second period of time, e.g. partly simultaneously with the flushing of the tube.

To determine the difference between the suction pressure and the ambient pressure, the pressure in the suction space could be measured by use of a pressure gauge of the kind known in the art. The pressure gauge could be located in the suction space, in the cover, in the flow path, e.g. fixed to the tube, or after the tube at the outlet thereof, or it may form part of the pump. Alternatively or additionally, an absolute pressure of the suction space could be determined by use of a reference space, i.e. an enclosed volume that has a pressure relative to which the suction pressure is measured. Also in this case, the pressure could be measured by a gauge located at the above-mentioned locations. The pressure could be determined while the pump is pumping, e.g. at a point of time shortly after the flushing. In that way, the pressure is determined at a point in time where the tube is relatively clean, and the pressure measuring can be more precise. If the tube is clean, the measuring can further be independent on the location of the pressure gauge. The pressure gauge could e.g. be located in the tube or at the outlet of the tube, and as the tube is clean, the measurement may form a relatively precise indication of the suction pressure at the cover. The pressure could in be measured continuously, e.g. by use of a continuous (analogue) measuring technique or by performing a plurality of subsequent measurements in a third period of time.

In one particular embodiment, the surface could be a skin surface of a living being, and the method could be applied for wound treatment in which fluids and exudates are removed from a wound via the tube.

In particular in combination with suction treatment of sensitive surfaces, e.g. in connection with wound treatment or in connection with treatment of a fragile surface which may suffer from a too high suction pressure, it may be advantageous to facilitate good pressure control. As aforementioned, a pressure gauge could be arranged in the suction space, in the flow path, or at the outlet of the tube. In accordance with the invention, pressure control may be handled by measuring the pressure either when there is a flow through the tube or when the pressure difference has been stabilized. In one embodiment, the pressure difference is established by suction via the pump, subsequently, the tube is flushed to remove fluid and objects such as exudates which may be stuck in the flow path. In a short period of time after the flushing is stopped, the pressure difference may increase where after the pressure difference is stabilized. The third period of time may in particular include a period in which the first tube is flushed or at least a period directly after the flushing of the first tube. By waiting too long time, there is a risk that new fluid, exudates etc. enter the hose and make the pressure fluctuate. To find the right time to perform the measurement, it may be desired to monitor the pressure over a longer period of time, e.g. over a period of time which covers at least one or both of the aforementioned first and second periods of time. When the pressure starts to fluctuate, the flushing may be started, and when the flushing is ended, the pressure may be monitored to determine when the pressure is stabilized. The pressure which is determined shortly after the stabile state is entered can be used to control the pump or similar control means for adjusting the pressure and thus for protecting the surface against too large suction forces.

In connection with removal of fluid from surfaces which may be sensitive to influence from ambient air, e.g. due to a contamination threat etc., the flushing fluid could be a sterilised gas, or a gas with specific characteristics e.g. with respect to humidity, content of oxygen etc. Such a fluid could be contained in a container, e.g. under pressure, until the fluid is released. If the flushing fluid is regular air from the ambient space, or as an extra precaution in combination with a gas having specific characteristics, the suction and flushing is coordinated to prevent entrance of the flushing fluid into the suction space. This may be achieved by constantly monitoring a pressure difference between the suction pressure and a pressure in the flow path and by ensuring that the pressure in the flow path is always lower than the suction pressure. In any sense, it may be ensured that the pressure in the suction space is always lower than the pressure in the ambient space since.

As it appears from the above description, the until now described method of removing fluid from a surface may also be seen as a method of measuring a pressure in a suction system or it may be seen as a method of avoiding occlusion in a suction tube.

In a second aspect, the invention provides a suction system for removing fluid from a wound, the system comprising:
a cover which is attachable to a circumference of the wound to form an enclosed suction space separate from an ambient space,
a first tube forming a flow path between an inlet located in the suction space and an outlet located outside the suction space,
a pump in fluid communication with the first tube and capable of providing a pressure difference between a suction pressure in the suction space and an ambient pressure in the ambient space, and
flushing means for providing an intermittent flow of air through at least a portion of the tube.

Pressure detection means may further be included. The pressure detection means is typically located remote from the wound, e.g. in connection with a pump head, a drive unit or a control unit.

The pressure difference between the suction pressure and the ambient pressure may be kept substantially fixed or it may alternate between different values of negative suction pressure. It is preferred to control the pressure difference within a range defined by an upper and a lower level.

Preferably, the valve may be operated while the pump is working in order not to increase pressure in the enclosure.

The operation of the valve may be automatic, activating the valve within regular intervals or the valve may be operated by a signal from the pressure detection means or a control unit. The valve may be activated when a predetermined level of pressure is reached, if the upper level is reached, the valve may be opened, if the lower level is reached, the valve may be closed and optionally a signal for the pump to increase negative pressure may be sent.

Pressure sensing means could be provided, or alternatively, the system may be adjusted to a satisfactory pressure level, and intermittently activation of the valve may serve to maintain the pressure level. In one embodiment pressure detection means may produce a signal to the control unit or to the first valve when a predetermined pressure level is reached.

The valve may be opened for a short period to flush the first tube, and a measurement of the pressure may be conducted immediately after, to achieve a correct pressure determination.

The system may further comprise a control unit, controlling one or more of the operations of the system, e.g. recording the pressure and adjusting the activity of the pump and/or the valve in order to maintain a predetermined negative pressure. In particular, the control unit may comprise control means operating the first valve to provide a flushing period of time with a flow of air through the tube. The control means may comprise data processing means programmed:
to sample a number of pressure measurements, each measurement being significant for a pressure measured in the flow path, in the suction space, or at the outlet, and
to select, based on a pattern of the sampling, a time to perform a flushing of the suction tube, and optionally
to select, based on the pattern, a control pressure and to use the control pressure to control operation of the pump.

The processing means could e.g. comprise a set of instructions in the form of software code whereby the control means becomes adapted to perform the abovementioned steps. As an example, the control means may evaluate the pressure, and when the pressure fluctuates, the flushing of the tube may be started. After the flushing, the pressure may be monitored again, and when the pressure is stabilised, the unit may evaluate if the pressure is suitable for the treatment in question and if necessary adjust the pump in accordance with a desired pressure and the pressure which is obtained. Alternatively, the first valve is operated manually.

The first valve is preferably located at a proximal end of the first tube, i.e. the end which is towards the cover. This location enables a gas flow through substantially the entire first tube when the first valve is activated, thus flushing the tube from any liquid strings.

Contrary to systems where a bleeding hole is established on the pump-enclosure conduit or in the cover, the first valve of the present invention may be operated to be in a closed or open position. Thus, the pump may be stopped when sufficient negative pressure is achieved, and started again when the negative pressure decreases. A bleeding hole is constantly open, and requires constant work of the pump in order to maintain negative pressure. Constant operation of the pump is power consuming and may be noisy and can be a nuisance to the patient.

The first valve may be connected to the first tube via a second tube. The tube attached to the wound enclosure may be divided into a fork of a first tube, connecting to the pump, and a second tube connecting to the first valve. Thus, the valve is not in direct contact with the first tube and the fluid passing through this, and the risk of clotting the valve may be reduced, as well as the valve may be closer to, or integrated in the rest of the system. In one embodiment, the first and second tubes provide flow paths with equal cross-sectional size, and in an alternative embodiment, the first tube provides a flow path with a smaller cross-sectional size than that provided by the second tube. This facilitates an increased flow speed in the first tube and may thus reduce the flushing time.

In order to avoid a decrease in the negative pressure in the enclosure when the first valve is opened, the first tube may be provided with a second valve between the enclosure and the first valve. This second valve is preferably closed when the first valve is open. The second valve may not be necessary if the pump is strong enough to maintain the pressure over the wound even when the first valve is open.

The second valve may be operated manually or by a signal from the first valve or the pressure detection means or control unit, but preferably the valve is opened/closed automatically by the pressure in the tube. In a preferred embodiment of the invention the second valve is open when the pressure in the enclosure is higher than the pressure in the tube. A suitable valve for such purpose may be a one-way valve, e.g. a duck valve.

The system may further comprise a canister for receiving the liquid, and separating the gas from the liquid of the wound fluid. The canister is preferably located in connection with the pump. The pump may comprise a pump head for pumping the fluid and a drive unit for driving the pump. The canister may be located before or after the pump. The canister may comprise absorbent material.

The system may further comprise separating means, for separating the fluid into gas and liquid.

The first valve may be connected to the first tube via a second tube.

The first tube may comprise a second valve located between the enclosure and the first valve.

The second valve may be closed when the first valve is open or the second valve may be open when the pressure in the enclosure is higher than the pressure in the tube.

A canister may be provided in the system, the canister may e.g. be located before or after the pump.

In a third aspect, the invention provides a method of providing tube flushing in a wound treatment system, the system comprising:
a cover which is attachable to a circumference of the wound site to form an enclosure which separates a suction space from an ambient space,
a first tube forming a flow path between an inlet located in the suction space and an outlet located outside the suction space, and
a pump in fluid communication with the first tube,
   the method comprising:
providing a pressure difference between a suction pressure in the suction space and an ambient pressure in the ambient space,
waiting a fourth period of time,
opening a passage for releasing an amount of air into the proximal end of the flow path to flush the tube,
waiting a fifth period of time,
closing the passage,
waiting a sixth period of time, and
measuring a pressure difference between the suction pressure and the ambient pressure.

The proximal end of the flow path is an end towards the suction space, i.e. in the beginning of the flow path when considered in the flow direction.

In a fourth aspect, the invention provides a method of controlling a pump in a wound treatment system, the system comprising:
a cover which is attachable to a circumference of the wound site to form an enclosure which separates a suction space from an ambient space,
a first tube forming a flow path between an inlet located in the suction space and an outlet located outside the suction space, and
a pump in fluid communication with the first tube
   the method comprising:
sampling a number of pressure measurements, each measurement being significant for a pressure measured in the flow path, in the suction space or at the outlet, and
selecting based on a pattern of the sampling, a time to perform a flushing of the suction tube.

The method may imply the step of selecting, based on the pattern, a limit pressure at which the pump is stopped or otherwise controlled to avoid a too large suction pressure.

The pattern of the sampling could comprise a number of subsequent pressure measurements and it could e.g. show that the pressure fluctuates. Such fluctuations may indicate that the tube is filled or partly filled with liquid or solid substances whereby it may be decided to flush the tube. Accordingly, the method may comprise the step of comparing the sampling pattern with a reference pattern, and to perform the flushing based on the comparison. In a corresponding manner, the pattern may be used for controlling the pump, i.e. when the pressure difference between the suction space and the ambient space increases, the pump capacity can be decreased.

In a fifth aspect, the invention provides a control unit for a wound treatment system, the system comprising:
a cover which is attachable to a circumference of the wound site to form an enclosure which separates a suction space from an ambient space,
a first tube forming a flow path between an inlet located in the suction space and an outlet located outside the suction space, and
a pump in fluid communication with the first tube,
   the control unit comprising data processing means programmed:
to sample a number of pressure measurements, each measurement being significant for a pressure measured in the flow path, in the suction space, or at the outlet, and
to select, based on a pattern of the sampling, a time to perform a flushing of the suction tube.

The system may be adapted to select, based on the pattern, a control pressure and using the control pressure to control operation of the pump, e.g. by reducing the speed or by stopping the pump when the suction pressure reaches a pressure limit.

In a sixth aspect, the invention provides a method of measuring a pressure in a suction system for removing fluid from a wound, the method comprising:
providing a cover which is attachable to a circumference of the wound to form an enclosed suction space separated from an ambient space,
providing a first tube forming a flow path between an inlet located in the suction space and an outlet located outside the suction space,
providing, by suction via the first tube, a pressure difference between a suction pressure in the suction space and an ambient pressure in the ambient space, and
flushing the first tube by providing, in a first period of time, a fluid flow through at least a major part of the tube,
   wherein the pressure is measured after the flushing of the tube, e.g. within a period of time which is in the range of, or shorter than, the period of time in which the first tube is flushed.

In a seventh aspect, the invention provides a method of avoiding occlusion in a suction tube for a suction system for removing fluid from a wound, the method comprising:
providing a cover which is attachable to a circumference of the wound to form an enclosed suction space separated from an ambient space,
providing a first tube forming a flow path between an inlet located in the suction space and an outlet located outside the suction space,
providing, by suction via the first tube, a pressure difference between a suction pressure in the suction space and an ambient pressure in the ambient space, and
flushing the first tube by providing, in a first period of time, a fluid flow through at least a major part of the tube.

Any of the third, fourth, fifth, sixth, and seventh aspects of the invention may be combined with the features disclosed with respect to the first and second aspects of the invention.

### BRIEF DESCRIPTION OF THE DRAWINGS

Embodiments of the invention is disclosed in details with reference to the drawings in which

FIG. 1 shows one embodiment of the invention,

FIG. 2 shows another embodiment of the invention and

FIG. 3 shows a detailed view of a part of one invention.

### DETAILED DESCRIPTION OF PREFERRED EMBODIMENTS

In the following, preferred embodiments of the invention will be described in further details with reference to the drawing.

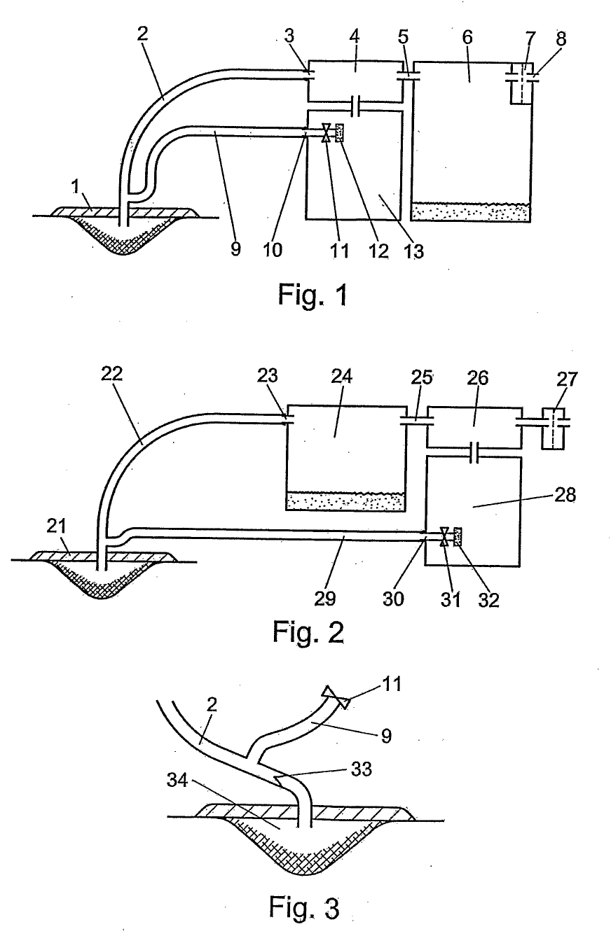

FIG. 1 shows a schematic drawing of the system. The wound cover (1) is sealing the wound to form an enclosure. Fluid in the form of air and liquid wound exudate are transported by suction through a first tube (2) by negative pressure created by the pump head (4) and drive unit (13). The first tube (2) may comprise several parts and different fittings, e.g. for releasable connection to the pump and/or the cover can be added.

The first tube (2) is connected to the pump head (4) by an inlet (3). The air and exudate are transported through the pump head (4) and by an outlet (5) to a canister (6). The canister can be any kind of volume suitable for collecting and/or separating air and liquid, such as a flexible bag.

In the canister (6), air and liquid are separated into two phases. The air will pass through the outlet (8) and the liquid will be retained in the canister (6). The canister may contain an absorbent component.

The outlet (8) of the canister may be provided with a filter (7) for removal of undesired components of the effluent gas.

The pump head (4) is connected to the drive unit (13). The pump head may be disposable.

A second tube (9) is connected to the first tube near the enclosure in one end and by an inlet (10) to a first valve (11) and filter (12) in the other end.

The first valve (11) will open and air will pass through the filter (12), second tube (9), first tube (2) and further through the rest of the system thus enabling free passage of gas through the first tube (2) and removal of any liquid strings.

Liquid strings in tubes/drains are contributing to an inaccuracy of the negative pressure in the system by inducing fluctuations in the pressure over the wound. These fluctuations are minimized by the use of the present system.

The first tube (2), canister (6), disposable pump head (4), second tube (9), valve (11), optionally filter (12) and filter (7) may be in the form of separate units or some or all of them may be integrated units.

The filter (7) can be a filter of any kind, a membrane or a combination of a filter and a membrane. The filter may be hydrophobic or lipophobic or able to retain bacteria. Several parameters can be determinative for opening of the valve (11) e.g. time, pressure, power consumption and others.

FIG. 2 shows another embodiment of the invention. The wound is sealed by a wound cover (21) and air and exudate are transported through a first tube (22) by negative pressure created by the pump head (26) and drive unit (28). The first tube (22) can comprise of several parts and different fittings can be added.

The exudate from the first tube (22) is collected in a canister (24). The first tube (22) is connected to the canister (24) through an inlet (23).

In the canister (24) gas and liquid are separated into two phases. The canister (24) can contain an absorbent component. The canister can be any kind of volume suitable for collecting/separate air and liquid.

The gas in the canister (24) is transported through an outlet (25) into the disposable pump head (26) by negative pressure.

A filter (27) can be placed after the disposable pump head (26), between the canister (24) and the disposable pump head (26) or both before and after the disposable pump head (26). The disposable pump head (26) is connected to the drive unit (28).

A second tube (29) is connected to the sealed wound or semi sealed wound (21) in one end and by an inlet (30) to a first valve (31) and filter (32) in the other end.

Now and then the first valve (31) will open and air will pass through the filter (32), second tube (29), first tube (22) and further through the rest of the system providing a first tube (22) without any liquid strings.

The first tube (22), canister (24), disposable pump head (26), second tube (29), first valve (31), filter (32) and filter (27) can be separate unit or some or all of them can be integrated units.

FIG. 3 discloses a detailed view of the two valves. The first tube (2) is connecting the enclosure (34) to the pump (not shown), and the second tube (9) is connecting the first valve (11) to the first tube near the enclosure (34). A second valve (33) is provided at the first tube, between the connection to the second tube (9) and the enclosure (34).

The second valve (33) may be open to fluids moving from the wound enclosure (34) towards the pump but not the other direction. When the first (11) valve is opened, the negative pressure decreases in the tubes (9, 2) and the valve (33) may close. Thus, the negative pressure over the wound will be maintained substantially unaffected by the opening of the first valve. When the first valve (11) is closed again, the negative pressure in the tubes (2, 9) increases, due to the work of the pump, and the second valve may therefore open again. In one embodiment, the second valve (33) operates based on the pressure and closes automatically when a pressure difference over the second valve (33) reaches a certain, typically adjustable, level.

1: A method for removing fluid from a surface, the method comprising:
providing a cover which is attachable to a circumference of the surface to form an enclosed suction space separated from an ambient space,
providing a first tube forming a flow path between an inlet located in the suction space and an outlet located outside the suction space,
providing, by suction via the first tube, a pressure difference between a suction pressure in the suction space and an ambient pressure in the ambient space, and
flushing the first tube by providing, in a first period of time, a flow of a flushing fluid through at least a major part of the tube.

2: A method according to claim 1, wherein the fluid flow is provided between the inlet and the outlet.

3: A method according to claim 1, wherein the tube is flushed by releasing an amount of the flushing fluid into the suction space or into the first tube.

4: A method according to claim 3, wherein the flush air is released into the first tube adjacent the inlet.

5: A method according to claim 3, wherein the flush air is provided via a first valve which is arranged to control fluid communication via a release passage between the ambient space and the suction space.

6: A method according to claim 1, wherein the pressure difference is provided by use of a suction pump which is activated in a second period of time, the second period of time overlying the first period of time.

7: A method according to claim 1, further comprising measuring a pressure in the suction space, in the flow path, or at the outlet.

8: A method according to claim 1, wherein the pressure is measured in a third period of time.

9: A method according to claim 6, wherein the third period of time overlaps the second period of time.

10: A method according to claim 6, wherein the third period of time overlaps the first period of time.

11: A method according to claim 8, wherein the first tube is flushed when a predetermined pressure or pressure pattern is determined.

12: A method according to claim 8, wherein the pressure is measured in a period of time after flushing of the tube is stopped.

13: A method according to claim 12, wherein the pressure difference is provided based on a pressure which is measured in a period of time between two subsequent flushing of the first tube.

14: A method according to claim 1, wherein the first period of time has a length which is sufficient to establish a flow through essentially an entire length of the first tube.

15: A method according to claim 1, applied for wound treatment.

16: A method according to claim 1, wherein the flushing and the suction is coordinated to prevent entrance of the flushing fluid in the suction space.

17: A method according to claim 1, wherein the flushing and the suction is coordinated to prevent equal pressure in the suction space and ambient space.

18: A suction system for removing fluid from a wound, the system comprising:
a cover which is attachable to a circumference of the wound to form an enclosed suction space separate from an ambient space,
a first tube forming a flow path between an inlet located in the suction space and an outlet located outside the suction space,
a pump in fluid communication with the first tube and capable of providing a pressure difference between a suction pressure in the suction space and an ambient pressure in the ambient space, and
flushing means comprising a first valve which provides a controllable fluid communication between the flow path and a source of flushing fluid for establishing an intermittent flow of air through at least a portion of the tube.

19: A system according to claim 18, wherein the valve provides controllable fluid communication with the ambient space.

20: A system according to claim 18, wherein the flushing means comprises a first valve which provides a controllable fluid communication between the suction space and the ambient space.

21: A system according to claim 18, comprising control means operating the first valve to provide a flushing period of time with a flow of air through the tube.

22: A system according to claim 21, wherein the control means comprises data processing means programmed:
to sample a number of measurements, each measurement being significant for a pressure measured in the flow path, in the suction space, or at the outlet, and
to select, based on a pattern of the sampling, a time to perform a flushing of the suction tube.

23: A system according to claim 22, wherein the control means is further adapted to select, based on the pattern, a control pressure and using the control pressure to control operation of the pump.

24: A system according to claim 21, wherein the flushing period of time has a length sufficient for the air to flow at least essentially through the flow path.

25: A system according to claim 18, comprising pressure detection means arranged to provide a signal in response to the pressure difference.

26: A system according to claim 25, wherein the flushing means is adapted to provide the flow of air in response to a predetermined pressure difference.

27: A system according to claim 18, wherein the flushing means is adapted to provide the flow of air while the pump is active.

28: A system according to claim 20, comprising a second tube connecting the first valve to the first tube.

29: A system according to claim 20, wherein the flushing means comprises a second valve which provides a controllable fluid communication between the suction space and the first valve.

30: A system according to claim 29, wherein the flushing means is adapted to close the second valve when the first valve is open.

31: A system according to claim 29, wherein the flushing means is adapted to open the second valve when the suction pressure is higher than a pressure in the first tube.

32: A system according to claim 29, wherein the second valve is a one way valve.

33: A system according to claim 18, comprising a canister for collection of fluid, the canister being located at a location between the suction space and the pump.

34: A system according to claim 18, comprising a canister for collection of fluid, the pump being located between the suction space and the canister so that the canister receives fluid which is pumped through the pump.

35: A method of providing tube flushing in a wound treatment system, the system comprising:
a cover which is attachable to a circumference of the wound site to form an enclosed suction space separate from an ambient space,
a first tube forming a flow path between an inlet located in the suction space and an outlet located outside the suction space, and
a pump in fluid communication with the first tube
the method comprising:
providing a pressure difference between a suction pressure in the suction space and an ambient pressure in the ambient space,
waiting a fourth period of time,
opening a passage for releasing an amount of air into the proximal end of the flow path to flush the tube,
waiting a fifth period of time,
closing the passage,
waiting a sixth period of time, and
measuring a pressure difference between the suction pressure and the ambient pressure.

36: A method of controlling a pump in a wound treatment system, the system comprising:
a cover which is attachable to a circumference of the wound site to form an enclosed suction space separate from an ambient space,
a first tube forming a flow path between an inlet located in the suction space and an outlet located outside the suction space, and
a pump in fluid communication with the first tube
the method comprising:
sampling a number of measurements, each measurement being significant for a pressure measured in the flow path, in the suction space or at the outlet, and
selecting, based on a pattern of the sampling, a time to perform a flushing of the suction tube.

37: A method according to claim 36, further comprising, selecting based on the pattern, a limit pressure at which the pump is stopped or pumping is reduced.

38: A control unit for a wound treatment system, the system comprising:
a cover which is attachable to a circumference of the wound site to form an enclosed suction space separate from an ambient space,
a first tube forming a flow path between an inlet located in the suction space and an outlet located outside the suction space, and
a pump in fluid communication with the first tube,
the control unit comprising data processing means programmed:
to sample a number of measurements, each measurement being significant for a pressure measured in the flow path, in the suction space, or at the outlet, and
to select, based on a pattern of the sampling, a time to perform a flushing of the suction tube.

39: A unit according to claim 38, further being adapted to select, based on the pattern, a limit pressure at which the pump is stopped or pumping is reduced

40: A method of measuring a pressure in a suction system for removing fluid from a wound, the method comprising:
providing a cover which is attachable to a circumference of the wound to form an enclosed suction space separated from an ambient space,
providing a first tube forming a flow path between an inlet located in the suction space and an outlet located outside the suction space,
providing, by suction via the first tube, a pressure difference between a suction pressure in the suction space and an ambient pressure in the ambient space, and
flushing the first tube by providing, in a first period of time, a fluid flow through at least a major part of the tube,
wherein the pressure is measured after the flushing of the tube.

41: A method of avoiding occlusion in a suction tube for a suction system for removing fluid from a wound, the method comprising:
providing a cover which is attachable to a circumference of the wound to form an enclosed suction space separated from an ambient space,
providing a first tube forming a flow path between an inlet located in the suction space and an outlet located outside the suction space,
providing, by suction via the first tube, a pressure difference between a suction pressure in the suction space and an ambient pressure in the ambient space, and
flushing the first tube by providing, in a first period of time, a fluid flow through at least a major part of the tube.
